(19) United States
(12) Plant Patent Application Publication
(10) Pub. No.: US 2025/0359497 P1
(43) Pub. Date: Nov. 20, 2025

(54) Osteospermum Plant Named 'KLEOE23096'

(50) ;

(71) Applicant: KLEMM + SOHN GMBH & CO. KG, Stuttgart (DE)

(72) Inventor: Maike Boxriker, Stuttgart (DE)

(73) Assignee: KLEMM + SOHN GMBH & CO. KG, Stuttgart (DE)

(21) Appl. No.: 18/665,552

(22) Filed: May 15, 2024

(51) Int. Cl. A01H 6/14; A01H 5/02

(52) U.S. Cl. CPC A01H 6/148; A01H 5/02
USPC PLT/360

(57) ABSTRACT

A new Osteospermum plant particularly distinguished by purple flowers, with an upright habit, is disclosed.

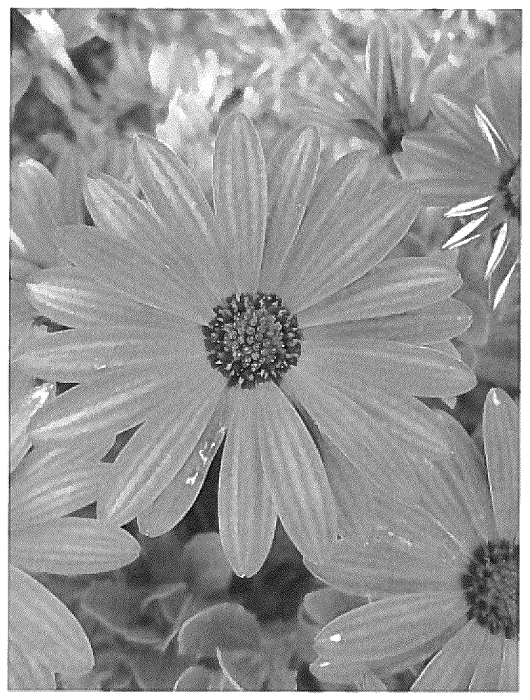

Genus and species: Osteospermum ecklonis Norl.

Variety denomination: ‘KLEOE23096’.

### BACKGROUND OF THE NEW PLANT

The present invention comprises a new and distinct variety of Osteospermum, botanically known as Osteospermum ecklonis Norl., and hereinafter referred to by the variety name ‘KLEOE23096’. ‘KLEOE23096’ originated from a cross-pollination conducted in summer 2015 in Stuttgart, Germany between the female Osteospermum variety ‘KLEO04110’ (U.S. Plant Pat. No. 18,272) and the proprietary male Osteospermum variety ‘OE-2014-1094’ (unpatented).

The seeds from the cross were collected and sown in Stuttgart, Germany in January 2016 and the plants were grown for evaluation. An individual plant designated ‘KLEOE23096’ was selected in June 2016 in Stuttgart, Germany. In June 2016 ‘KLEOE23096’ was first vegetatively propagated by vegetative terminal cuttings in Stuttgart, Germany. ‘KLEOE23096’ was found to reproduce true to type in successive generations of asexual propagation via vegetative cuttings.

### SUMMARY

The following are the most outstanding and distinguishing characteristics of this new variety when grown under normal horticultural practices in Stuttgart, Germany.
1. Purple flowers; and
2. Upright habit.

### DESCRIPTION OF THE PHOTOGRAPH

This new Osteospermum plant is illustrated by the accompanying photograph which shows a close-up of the foliage, buds, and mature flower. The photograph is of a 17-week-old plant grown outdoors in Stuttgart, Germany in July 2023. The colors shown are as true as can be reasonably obtained by conventional photographic procedures.

### DESCRIPTION OF THE NEW VARIETY

The following detailed descriptions set forth the distinctive characteristics of ‘KLEOE23096’. The data which define these characteristics were collected in Stuttgart Germany in April 2024 on plants potted in October 2023. References are to The R.H.S. Colour Chart of The Royal Horticultural Society of London (R.H.S.), 5^{th} edition (2007).
Classification:
   Family.—Asteraceae.
   Botanical.—Osteospermum ecklonis Norl.
   Common.—Osteospermum.
   Designation.—‘KLEOE23096’.
Parentage:
   Female parent.—The female Osteospermum variety ‘KLEO04110’ (U.S. Plant Pat. No. 18,272).
   Male parent.—The proprietary male Osteospermum variety ‘OE-2014-1094’ (unpatented).
Plant:
   Form and habit.—Herbaceous perennial but usually cultivated as an annual; not frost resistant.
   Growth habit.—Upright.
   Branching habit.—Normal.
   Height (from top of soil, including inflorescence).—16.0 cm.
   Width (including inflorescences).—20.0 cm.
   Propagation.—Vegetative cuttings.
   Time to produce a finished flowering plant.—10 to 17 weeks.
   Time to initiate and develop roots.—4 weeks.
   Root description.—Fine; RHS 155A (white).
Leaves:
   Arrangement.—Alternate.
   Shape.—Obovate.
   Apex.—Acute.
   Base.—Acute.
   Margin.—Entire.
   Immature color.—Upper surface: RHS 144A. Lower surface: RHS 144A.
   Mature color.—Upper surface: RHS 137A. Lower surface: RHS 137C.
   Length.—3.1 cm.
   Width.—1.2 cm.
   Texture.—Upper surface: Leathery. Lower surface: Leathery with fine hairs.
   Petioles.—Absent.
   Venation pattern.—Absent.
   Venation color.—Upper surface: RHS 144A. Lower surface: RHS 144A.
Stems:
   Total number of branches per plant (calculated at the base of the plant).—4.
   Length.—15.0 cm.
   Diameter.—0.3 cm.
   Internode length.—0.9 cm.
   Color.—RHS 144B.
   Texture.—Smooth.
   Anthocyanin.—Absent.
Buds:
   Shape.—Round.
   Length.—1.1 cm.
   Diameter.—0.8 cm.
   Color (at tight bud just before the ray florets unfold).—RHS 137C.
Inflorescence:
   Type.—Composite, single.
   Blooming habit.—Upright, with longer flower stems.
   Quantity of inflorescences per plant.—32.
   Lastingness of the inflorescences on the plant.—10 to 15 days.
   Fragrance.—Present.
   Inflorescence diameter.—5.4 cm.
   Inflorescence depth.—1.5 cm.
   Disc diameter.—1.3 cm.
Disc floret:
   Quantity per inflorescence.—Around 50.
   Shape.—Tubular.
   Tube color.—Closed: RHS 79A. Mature: RHS 79A.
   Length.—0.7 cm.
   Diameter (at apex).—0.3 cm.
   Apex.—Acute.
   Apex color.—RHS 79A.
   Base.—0.1 cm.
   Base color.—RHS 157A.
   Margin.—Entire.
Ray floret:
   Quantity per inflorescence.—21.
   Shape.—Oblanceolate.
   Color.—Upper surface: RHS N78A with a ring (RHS N81A) that is absent or very narrow and elongated stripes (RHS 84D) that are absent or very weak but may appear in the summertime when grown outdoors. Lower surface: RHS N78C.
   Length.—2.4 cm.
   Width.—0.5 cm.
   Apex.—Obtuse.
   Base.—Acute.
   Margin.—Entire.
   Texture.—Upper surface: Smooth. Lower surface: Smooth.
   Peduncle.—Length: 5.3 cm. Diameter: 0.1 cm. Texture: Smooth with hairs. Color: RHS 144A.
Phyllaries:
   Texture.—Smooth with fine hairs.
   Color.—RHS 138A.
   Arrangement.—Single.
   Observed quantity per plant.—21.
   Shape.—Lanceolate.
   Color.—Upper surface: RHS 138A. Lower surface: RHS 138B.
   Length.—2.2 cm.
   Width.—0.2 cm.
   Apex.—Acute.
   Base.—Obtuse.
   Margin.—Entire.
   Texture.—Upper surface: Smooth with fine hairs. Lower surface: Smooth.
Reproductive organs:
   Androecium.—Location: On the disc floret. Stamens: Quantity: Numerous, approximately 100 . Shape: Filamentous. Filament length: 0.2 cm. Filament diameter: Less than 0.1 cm.
   Anther.—Shape: Lanceolate. Color: RHS 79A. Length: 0.1 cm. Diameter: Less than 0.1 cm.
   Pollen.—Color: RHS 14A. Amount: Abundant.
   Gynoecium.—Location: On the base of the petals (ray floret). Pistils: Number: Numerous, approximately 30. Length: 0.2 cm. Diameter: Less than 0.1 cm Stigma: Color: RHS 79A. Shape: Filamentous. Length: 0.2 cm. Diameter: Less than 0.1 cm. Style: Color: RHS 79A. Length: 0.1 cm. Diameter: Less than 0.1 cm.
Fruit and seed set: None observed.

Disease and pest/insect resistance: No disease and pest/insect resistance observed.

### COMPARISON WITH PARENTAL LINES AND KNOWN VARIETY

‘KLEOE23096’ is a distinct variety of Osteospermum and is distinguished from its parents as described in Table 1.

TABLE 1
Comparison with Parental Lines

| Characteristic | ‘KLEOE23096’ | Female Parent | Male Parent |
|---|---|---|---|
| Flower filling | Single | Double | Single |
| Flower color | Purple | Red to Purple | Purple |
| Habit | Upright | Upright, compact and | Compact |
| | | mounded | |

‘KLEOE23096’ is most similar to the commercial Osteospermum variety ‘KLEOE23094’ (unpatented). Differences between the varieties are described in Table 2.

TABLE 2
Comparison with Similar Variety

| Characteristic | ‘KLEOE23096’ | ‘KLEOE23094’ |
|---|---|---|
| Habit | Upright | Compact |
| Earliness | Earliness is later than | Earlier than |
| | ‘KLEOE23094’ | ‘KLEOE23096’ |

I claim:

1. A new and distinct cultivar of Osteospermum Plant Named ‘KLEOE23096’ as described and illustrated herein.
